(19) United States
(12) Patent Application Publication
(10) Pub. No.: US 2024/0151705 A1
(43) Pub. Date: May 9, 2024

(54) SOIL SENSOR AND METHODS THEREOF

(71) Applicant: HGCI, Inc., Las Vegas, NV (US)

(72) Inventors: Dengke Cai, Camas, WA (US); Wen Zhang, Ridgefield, WA (US); Yinlin Li, Camas, WA (US)

(21) Appl. No.: 18/505,920

(22) Filed: Nov. 9, 2023

Related U.S. Application Data
(60) Provisional application No. 63/424,106, filed on Nov. 9, 2022.

(51) Int. Cl. G01N 33/24; G01K 1/024; G01K 3/00; G01N 27/04

(52) U.S. Cl. CPC G01N 33/246; G01K 1/024; G01K 3/005; G01N 27/04; G01N 2033/245

(57) ABSTRACT

A soil sensor for a horticultural environment is provided. The soil sensor includes at least one probe, a sensing module, and an onboard controller. The at least one probe is configured for insertion into a soil substrate. The sensing module is associated with the at least one probe and is configured to detect an environmental parameter of the soil substrate via the at least one probe. The onboard controller is in signal communication with the sensing module and is configured to determine a value of the environmental parameter from the measured environmental parameter.

### REFERENCE TO RELATED APPLICATION

This application claims priority of U.S. provisional patent application Ser. No. 63/424,106 filed Nov. 9, 2022, and hereby incorporates this patent application by reference herein in its entirety.

### TECHNICAL FIELD

The apparatus described below generally relates to a soil sensor for a horticultural environment. In particular, the soil sensor can be configured to detect an environmental parameter of a soil substrate to facilitate notification to a user when the environmental parameter is out of range.

### BACKGROUND

When plants are grown in soil, certain environmental parameters of the soil, such as volumetric water content, electrical conductivity and temperature, can affect the viability and growth of the plants. A soil sensor that is configured to detect only one of these environmental parameters can be installed in the soil to allow a user to intervene when the individual environmental parameter is outside of a desired range.

### BRIEF DESCRIPTION OF THE DRAWINGS

Various embodiments will become better understood with regard to the following description, appended claims and accompanying drawings wherein:

FIG. 1 is an upper rear isometric view depicting a soil sensor and a cable;

FIG. 2 is a lower front isometric view depicting the soil sensor and cable of FIG. 1;

FIG. 3 is a schematic view of the soil sensor of FIGS. 1 and 2, the soil sensor including an onboard controller and a volumetric water content sensing module; and

FIG. 4 is a schematic view of the onboard controller and the volumetric water content of FIG. 3.

### DETAILED DESCRIPTION

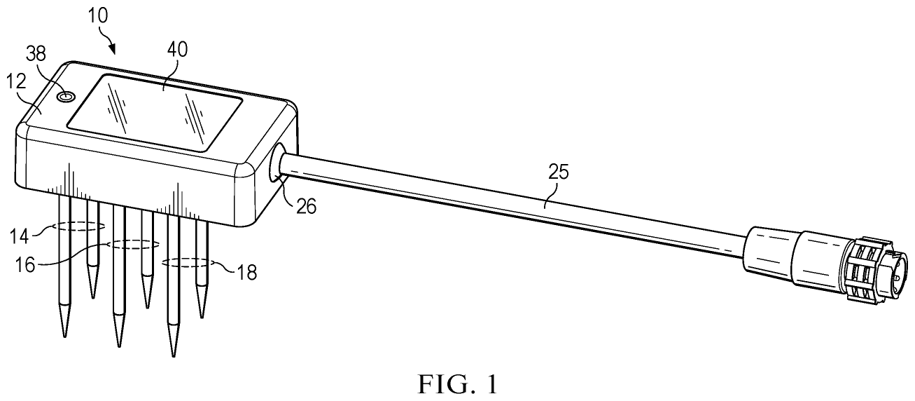

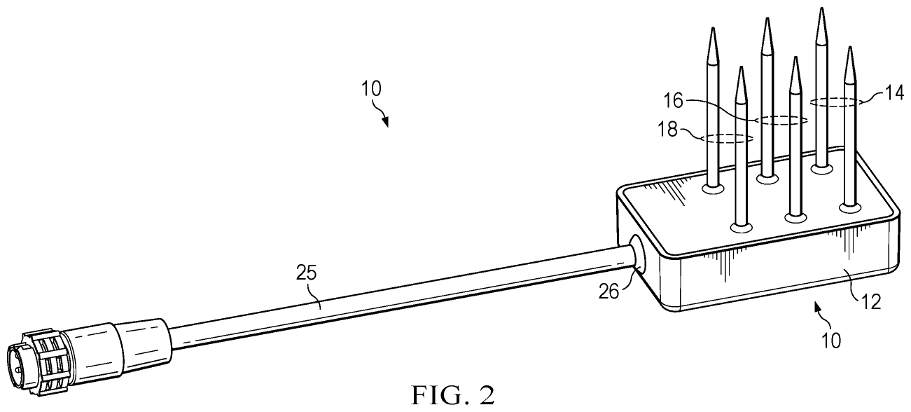

Embodiments are hereinafter described in detail in connection with the views and examples of FIGS. 1-4, wherein like numbers indicate the same or corresponding elements throughout the views. A soil sensor 10 is generally depicted in FIGS. 1 and 2 and is shown to include a housing 12 and first, second, and third pairs of probes 14, 16, 18 that extend outwardly from the housing 12. Each of the respective pairs of probes 14, 16, 18 can be configured to facilitate detection of a different environmental parameter of a soil substrate. In one embodiment, the first pair of probes 14 can facilitate detection of volumetric water content of a soil substrate, the second pair of probes can facilitate detection of electrical conductivity of the soil substrate, and the third pair of probes 18 can facilitate detection of a temperature of the soil substrate. To facilitate detection of the environmental parameters of the soil substrate, the soil sensor 10 can be installed into a soil substrate by inserting each of the probes 14, 16, 18 into a soil substrate such that each of the probes 14, 16, 18 extends at least partially beneath the soil substrate surface. The housing 12 can remain above the soil substrate surface when the soil sensor 10 is fully installed. Each of the probes 14, 16, 18 can be formed of a conductive material, such as aluminum or stainless steel, and can have an elongated shape that terminates at a point that allows for easy insertion of the probes 14, 16, 18 into the soil substrate. As will be described in further detail below, the soil sensor can be configured to generate one or more notifications to a user that allows the user to monitor the environmental parameters and take appropriate action when the environmental parameter(s) is/are out of range.

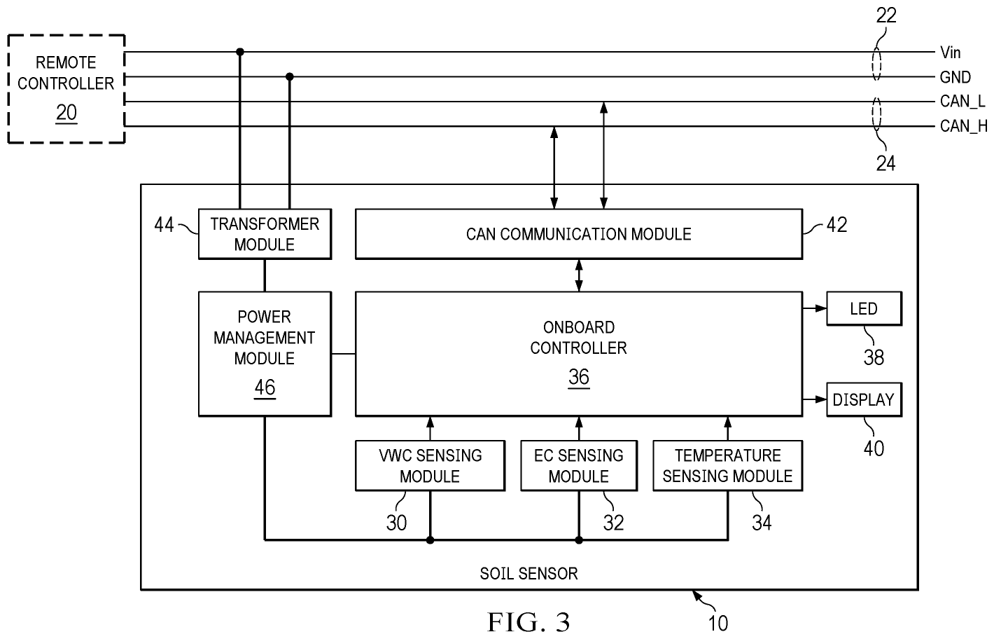

Referring now to FIG. 3, a schematic view of the soil sensor 10 and a remote controller 20 is illustrated and will now be described. The soil sensor 10 can be powered by a power bus 22 that is electrically coupled with the remote controller 20 and receives power therefrom. The power bus 22 can be powered by input power, typically 120 VAC, that is used to power the remote controller 20. In one embodiment, the remote controller 20 can include an internal transformer (not shown) that converts the input power received by the remote controller 20 into rated power, typically DC power, for powering the soil sensor 10. It is to be appreciated that the soil sensor 10 can additionally or alternatively be powered by an external power source that is routed directly to the soil sensor 10 and thus bypasses the remote controller 20. The soil sensor 10 can be communicatively coupled with the remote controller 20 via a controller area network (CAN) communication bus 24 such that the remote controller 20 can communicate with the soil sensor 10 via a CAN protocol, as will be described in further detail below.

A cable 25 can be provided that houses both the power bus 22 and the CAN bus 24 and is plugged into each of the soil sensor 10 and the remote controller 20. The soil sensor 10 can include an electrical connector 26 (FIGS. 1 and 2) that allows for electrical and communicative coupling of the soil sensor 10 to the cable 25. The electrical connector 26 can be any of a variety of connection types, such as, for example, a CNT-13 type connector, a Wieland-type connector, an RJ-45 connector, a push-pull connector, or a quick-lock connector. It is to be appreciated that the remote controller 20 can also include an electrical connector (not shown) that allows for electrical and communicative coupling of the remote controller 20 to the cable. It is to be appreciated that although only one soil sensor 10 is shown to be connected to the remote controller 20, the remote controller 20 can support a plurality of soil sensors that are connected together via the cable 25 (i.e., daisy chained together) and are powered by, and communicate with, the remote controller 20 in a similar manner as described herein for the soil sensor 10. An example of the remote controller 20 is illustrated and described in PCT Pub. No. WO2022/266475 which is hereby incorporated by reference herein in its entirety.

Still referring to FIG. 3, the soil sensor 10 can include a volumetric water content sensing module 30 (hereinafter “VWC sensing module 30”), an electrical conductivity sensor 32 (hereinafter “EC sensing module 32”) and a temperature sensing module 34. An onboard controller 36 can be in signal communication with each of the VWC sensing module 30, the EC sensing module 32, and the temperature sensing module 34 and can be configured to process sensor data received therefrom as described in more detail below. The onboard controller 36 may be embodied as any type of processor capable of performing the functions described herein. For example, the onboard controller 36 may be embodied as a single or multi-core processor, a digital signal processor, a microcontroller, a general purpose central processing unit (CPU), a reduced instruction set computer (RISC) processor, a processor having a pipeline, a complex instruction set computer (CISC) processor, an application specific integrated circuit (ASIC), a programmable logic device (PLD), a field programmable gate array (FPGA), or other processor or processing/controlling circuit or controller.

The VWC sensing module 30 can be configured to facilitate detection of the amount of volumetric water content of the soil substrate. The VWC sensing module 30 can be associated with the first pair of probes 14 (FIGS. 1 and 2). The VWC sensing module 30 can be configured to generate a reference signal that is imparted onto both of the first probes 14 and that propagates through the soil substrate. In one embodiment, the reference signal can be a fast rise step voltage pulse that propagates as an electromagnetic wave through the soil substrate. The propagation of the reference signal through the soil substrate induces a reflected signal that is received at the first probes 14. The VWC sensing module 30 can measure the difference between the reflected signal and the reference signal and can provide that measurement as an output signal to the onboard controller 36. In one embodiment, the VWC sensing module 30 can measure the phase difference between the reference signal and the reflected signal and can generate a pulsed output signal that indicates the phase difference to the onboard controller 36.

The onboard controller 36 can then use the output signal from the VWC sensing module 30 to determine an amount of volumetric water content of the soil substrate. In one embodiment, the onboard controller 36 can utilize a lookup table that maps different output signal magnitudes to specific values of volumetric water content. In some instances, the onboard controller 36 can average the values of volumetric water content obtained from different output signals from the VWC sensing module 30 over a discrete time period to account for erroneous measurements that might be present on some of output signals during that discrete time period.

The onboard controller 36 can be in signal communication with an LED 38 that is disposed on the housing 12 (FIG. 1). When the value of the volumetric water content is within a desired range, the onboard controller 36 can cause the LED 38 to be illuminated with a colored light, such as a green light, that indicates to a user that the volumetric water content of the soil substrate is acceptable. When the value of the volumetric water content is outside of a desired range, the onboard controller 36 can cause the LED 38 to be illuminated with a different colored light, such as a red light, that indicates to a user that the volumetric water content of the soil substrate is not acceptable.

The onboard controller 36 can also be in signal communication with a display 40 that is present on an opposite side of the housing 12 as the probes 14, 16, 18 (FIG. 1). The onboard controller 36 can cause the value of the volumetric water content to be displayed on the display 40 for presentation to the user. In one embodiment, the volumetric water content can be updated in real time. The display 40 can be an LCD screen, a segmented display or any other display type that is capable of presenting the volumetric water content value to a user.

The onboard controller 36 can be in signal communication with a CAN communication module 42 that is communicatively coupled with the CAN bus 24 and is configured to communicate with the remote controller 20 using a CAN architecture. The CAN architecture can facilitate bidirectional communication between the soil sensor 10 and the remote controller 20 and between the soil sensors themselves. The remote controller 20 can poll the soil sensor 10 for volumetric water content data that might include the volumetric water content value and an indication of whether the volumetric water content is outside of a predefined range. The onboard controller 36 can respond accordingly with a response message that is transmitted to the remote controller 20 and that includes the volumetric water content data requested by the remote controller 20. In response, the remote controller 20 can cause the volumetric water content value to be displayed on a native screen (not shown) and/or can generate an alarm for a user if the volumetric water content is outside of a predefined range.

The CAN architecture can also allow the remote controller 20 to detect a problem with the health of the soil sensor 10. If the remote controller 20 detects a problem with the health of one or more of the soil sensors 10 (e.g., based on the response message from the soil sensor 10), such as a communication problem or as a result of a fault code transmitted to the CAN bus by the remote controller 20, the remote controller 20 can notify a user of the problematic soil sensor by activating an indicator (e.g., a light or an audible sound) on the problematic soil sensor, activating an indicator on a surrounding soil sensor (e.g., intermittently illuminating an indicator on an adjacent soil sensor to the problematic soil sensor (e.g., an immediately upstream or downstream sensor)), and/or displaying the unique ID of the problematic soil sensor on the native display.

Still referring to FIG. 3, the EC sensing module 32 can be configured to facilitate detection of the electrical conductivity of the soil substrate. The EC sensing module 32 can be associated with the second pair of probes 16 (FIGS. 1 and 2). The EC sensing module 32 can be configured to generate an EC reference signal that is imparted onto both of the second probes 16 and that propagates through the soil substrate. The propagation of the EC reference signal through the soil substrate induces a reflected signal that is received at the second probes 16. The EC sensing module 32 can measure the difference between the EC reflected signal and the EC reference signal and can provide that measurement as an output signal to the onboard controller 36 that is indicative of the electrical conductivity of the soil substrate. In one embodiment, the EC sensing module 32 can measure the phase difference between the EC reference signal and the EC reflected signal and can generate a pulsed output signal that indicates the phase difference to the onboard controller 36.

The onboard controller 36 can then use the output signal from the EC sensing module 32 to determine an amount of electrical conductivity of the soil substrate. In one embodiment, the onboard controller 36 can utilize a lookup table that maps different output signal magnitudes to specific values of electrical conductivity. In some instances, the onboard controller 36 can average the values of electrical conductivity obtained from different output signals from the EC sensing module 32 over a discrete time period to account for erroneous measurements that might be present on some of the output signals during that discrete time period.

When the value of the electrical conductivity is within a desired range, the onboard controller 36 can cause the LED 38 to be illuminated with a colored light, such as a green light, that indicates to a user that the electrical conductivity of the soil substrate is acceptable. When the value of the electrical conductivity is outside of a desired range, the onboard controller 36 can cause the LED 38 to be illuminated with a different colored light, such as a red light, that indicates to a user that the electrical conductivity of the soil substrate is not acceptable.

The onboard controller 36 can additionally or alternatively cause the value of the electrical conductivity to be displayed on the display 40 for presentation to the user. In one embodiment, the electrical conductivity can be updated in real time. The onboard controller 36 can transmit a response message to the remote controller 20 via the CAN bus 24 that includes electrical conductivity data that the remote controller 20 can use in a similar manner as described above with respect to the volumetric water content data.

Still referring to FIG. 3, the temperature sensing module 34 can be configured to facilitate detection of the temperature of the soil substrate. The temperature sensing module 34 can be associated with the third pair of probes 18 (FIGS. 1 and 2). The temperature sensing module 34 can be configured to generate a temperature reference signal that is imparted onto both of the third probes 18 and that propagates through the soil substrate. The propagation of the temperature reference signal through the soil substrate induces a reflected signal that is received at the third probes 18. The temperature sensing module 34 can measure the difference between the EC reflected signal and the EC reference signal and can provide that measurement as an output signal to the onboard controller 36 that is indicative of the temperature of the soil substrate. In one embodiment, the temperature sensing module 34 can measure the phase difference between the temperature reference signal and the temperature reflected signal and can generate a pulsed output signal that indicates the phase difference to the onboard controller 36.

The onboard controller 36 can then use the output signal from the temperature sensing module 34 to determine the temperature of the soil substrate. In one embodiment, the onboard controller 36 can utilize a lookup table that maps different output signal magnitudes to specific temperature values. In some instances, the onboard controller 36 can average the temperature values obtained from different output signals from the temperature sensing module 34 over a discrete time period to account for erroneous measurements that might be present on some of the output signals during that discrete time period.

When the value of the temperature is within a desired range, the onboard controller 36 can cause the LED 38 to be illuminated with a colored light, such as a green light, that indicates to a user that the temperature of the soil substrate is acceptable. When the value of the temperature is outside of a desired range, the onboard controller 36 can cause the LED 38 to be illuminated with a different colored light, such as a red light, that indicates to a user that the temperature of the soil substrate is not acceptable.

The onboard controller 36 can additionally or alternatively cause the value of the temperature to be displayed on the display 40 for presentation to the user. In one embodiment, the temperature can be updated in real time. The onboard controller 36 can transmit a response message to the remote controller 20 via the CAN bus 24 that includes temperature data that the remote controller 20 can use in a similar manner as described above with respect to the volumetric water content data. It is to be appreciated that although a VWC sensing module, an EC sensing module, and a temperature sensing module are described herein for detecting the respective environmental parameters described herein (e.g., the volumetric water content, the electrical conductivity, and the temperature) one of those sensing modules or different combinations of those sensing modules can be incorporated into the soil sensor 10. It is also to be appreciated that other sensing modules can be incorporated into the soil sensor 10 for detecting other types of environmental parameters of a soil substrate.

Still referring to FIG. 3, the soil sensor 10 can include a transformer module 44 that is electrically connected on one side to the power bus 22 and at the other side to each of the VWC sensing module 30, the EC sensing module 32, the temperature sensing module 34, and the onboard controller 36 to facilitate powering thereof from the power bus 22. The transformer module 44 can be configured to transform the power from the power bus 22 into usable power for powering the VWC sensing module 30, the EC sensing module 32, the temperature sensing module 34, and the onboard controller 36. In one embodiment, the transformer module 44 can be configured to generate different DC voltages (e.g., 5 VDC, 12 VDC, 15 VDC) for powering the VWC sensing module 30, the EC sensing module 32, the temperature sensing module 34, and the onboard controller 36. In one embodiment, the transformer module 44 can comprise an isolated DC/DC converter. A power management module 46 can be provided downstream of the transformer module 44 and can be configured to condition the power signal delivered from the transformer module 44 before it reaches the VWC sensing module 30, the EC sensing module 32, the temperature sensing module 34, and the onboard controller 36.

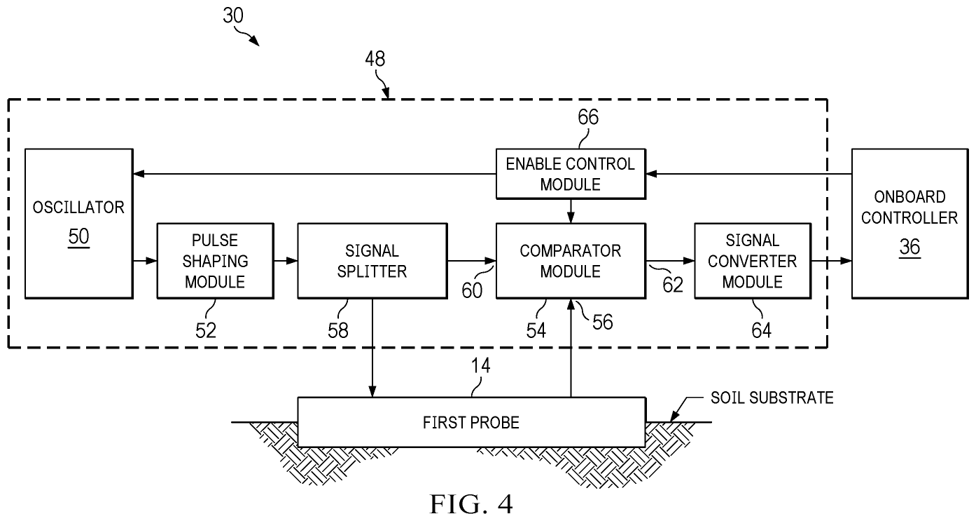

Referring now to FIG. 4, a schematic view of the VWC sensing module 30 is illustrated and will now be described. The VWC sensing module 30 can include a sensing circuit 48 that includes a signal generator in the form of an oscillator 50 that is in signal communication with one of the first probes 14. The oscillator 50 is configured to generate the reference signal that is imparted onto the first probe 14 and that propagates through the soil substrate. The reference signal from the oscillator 50 can be a pulsed signal that has a signal frequency, such as a 100 MHz fast rise step voltage pulse that propagates as an electromagnetic wave through the soil substrate. The reference signal from the oscillator 50 can be provided through a pulse shaping module 52 that is configured to enhance the rising and falling edge of the pulsed signal which can contribute to the accuracy of the comparison with the reflected signal and therefore can enhance the accuracy of the measurement of the difference between the reflected signal and the reference signal.

A comparator module 54 can be provided that is configured to compare the reference signal and the reflected signal and generate the output signal based upon the comparison of the reference signal and the reflected signal. The comparator module 54 can include a first input 56 that is in signal communication with the first probe 14 and is configured to receive the reflected signal from the first probe 14. The reference signal that is transmitted to the first probe 14 from the oscillator 50 can also be simultaneously provided to the comparator module 54 by a signal splitter 58 in order to facilitate comparison of the reference signal and the reflected signal at the comparator module 54. The signal splitter 58 can be in signal communication with the oscillator 50 (via the pulse shaping module 52), the first probe 14, and a second input 60 of the comparator module 54. The oscillator 50 can therefore be in signal communication with each of the first probe 14 and the first input 56 via the pulse shaping module 52 and signal splitter 58.

The signal splitter 58 can receive the reference signal from the oscillator 50 and can cause the reference signal to be duplicated and routed to each of the first probe 14 and the second input 60. The reference signal used by the comparator module 54 for comparison with the reflected signal is therefore consistent with, and in some cases exactly the same as, the reference signal that is propagated through the soil which can enhance the accuracy of the comparison. This redundancy can ensure that the difference between the reference signal and the reflected signal at the first probe 14 is accurately reflected at the comparator module 54 which can enhance the accuracy of the detection of the volumetric water content from the reference signal and the reflected signal.

The comparator module 54 can receive the reflected signal at the first input 56 and the reference signal at the second input 60 and can compare the two signals to measure the differences therebetween. The measurement can be provided to the onboard controller 36 as the output signal via an output 62 that is in signal communication with the onboard controller 36. The output signal can be routed through a signal converter module 64 that converts the output signal into a format that is capable of being utilized by the onboard controller 36, such as for example, from a digital signal into an analog signal. The signal converter module 64 can also be configured to condition the output signal to enhance the precision of the output signal. The onboard controller 36 can determine a value of the volumetric water content in the soil substrate that can be used for one or more of the purposes described above.

In one embodiment, the comparator module 54 can include an XOR gate that receives the reference signal and the reflected signal at its inputs and delivers the output signal from its outputs. Generally speaking, the XOR gate will generate a high output signal (e.g., a logical one) when the input signals are opposite from each other (e.g., one input is high and the other input is low). When the reference signal propagates through the soil, the volumetric water content in the soil substrate causes a phase difference between the reflected signal and the reference signal that is substantially proportional to the amount of volumetric water content in the soil substrate. When the reference signal and the reflected signal are routed through the XOR gate, the phase difference between the reference signal and the reflected signal generates a pulse on the output signal. The pulse width of the pulse can be proportional to the amount of volumetric water content of the soil. The output signal can be routed through the signal converter module 64 which converts the pulse into a different format, such as an analog signal, that is representative of the pulse width of the output signal and can be used by the onboard controller 36 to calculate the value of the volumetric water content of the soil substrate as a function of the pulse width of the output signal.

Still referring to FIG. 4, the sensing circuit 48 can also include an enable control module 66 that is in signal communication with the oscillator 50 and the comparator module 54 and is configured to selectively activate the oscillator 50 and the comparator module 54. When the oscillator 50 and the comparator module 54 are activated, the sensing circuit 48 is operable to detect the volumetric water content. However, when the oscillator 50 and the comparator module 54 are deactivated, the sensing circuit 48 is inoperable to detect the volumetric water content.

The onboard controller 36 can be in signal communication with the enable control module 66 and can be configured to generate a control signal for the enable control module 66 that causes the enable control module 66 to selectively activate the oscillator 50 and the comparator module 54, and thus the sensing circuit 48, in response to the control signal. The onboard controller 36 can coordinate the activation and deactivation of the sensing circuit 48 via the control signal to allow for the detection of the volumetric water content to only occur periodically such that the sensing circuit 48 is effectively in a sleep mode when the oscillator 50 and the comparator module 54 are deactivated which can conserve power and can extend the overall useful life of the various components of the sensing circuit 48. It is to be appreciated that the principles and details described above with respect to the sensing circuit 48 can additionally or alternatively be applied to the EC sensing module 32, the temperature sensing module 34, or any of a variety of other sensing modules that are incorporated into the soil sensor 10.

The foregoing description of embodiments and examples has been presented for purposes of illustration and description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein but can be employed in any number of applications and equivalent devices by those of ordinary skill in the art. Rather, it is hereby intended that the scope be defined by the claims appended hereto. Also, for any methods claimed and/or described, regardless of whether the method is described in conjunction with a flow diagram, it should be understood that unless otherwise specified or required by context, any explicit or implicit ordering of steps performed in the execution of a method does not imply that those steps must be performed in the order presented and may be performed in a different order or in parallel.

What is claimed is:

1. A soil sensor for a horticultural environment, the soil sensor comprising:
at least one probe configured for insertion into a soil substrate;
a sensing module associated with the at least one probe and configured to measure an environmental parameter of the soil substrate via the at least one probe;
an onboard controller in signal communication with the sensing module and configured to determine a value of the environmental parameter from the measured environmental parameter; and
a CAN communication module in signal communication with the onboard controller and configured to facilitate bidirectional communication with a remote controller via a controller area network architecture, wherein the onboard controller is configured to transmit the value of the environmental parameter to the remote controller via the CAN communication module.

2. The soil sensor of claim 1 wherein:
the environmental parameter comprises volumetric water content; and
the sensing module comprises a volumetric water content sensing module that is configured to measure the volumetric water content of the soil substrate.

3. The soil sensor of claim 1 wherein:
the environmental parameter comprises electrical conductivity; and
the sensing module comprises an electrical conductivity sensing module that is configured to measure the electrical conductivity of the soil substrate.

4. The soil sensor of claim 1 wherein:
the environmental parameter comprises temperature; and
the sensing module comprises a temperature sensing module that is configured to measure the temperature of the soil substrate.

5. The soil sensor of claim 1 further comprising a display screen configured to display information about the environmental parameter.

6. The soil sensor of claim 1 further comprising an indicator.

7. The soil sensor of claim 1 further comprising a communication port that is configured for attachment to a communication cable that facilitates wired communication with the remote controller.

8. A soil sensor for a horticultural environment, the soil sensor comprising:
at least one probe configured for insertion into a soil substrate and configured to facilitate delivery of a reference signal into the soil substrate and subsequent receipt of a reflected signal that is generated as a function of propagation of the reference signal through the soil substrate; and
a sensing module associated with the at least one probe and comprising a sensing circuit configured to sense an environmental parameter of the soil substrate via the at least one probe, the sensing circuit comprising:
a signal generator in signal communication with the at least one probe and configured to generate the reference signal at a frequency to facilitate delivery of the reference signal to the soil substrate; and
a comparator module comprising a first input, a second input, and an output, wherein:
the first input is in signal communication with the signal generator such that the first input receives the reference signal from the signal generator simultaneously with the at least one probe;
the second input is in signal communication with the at least one probe and is configured to receive the reflected signal from the at least one probe;
the comparator module is configured to compare the reference signal and the reflected signal and generate an output signal at the output based upon the comparison of the reference signal and the reflected signal; and
the output signal facilitates determination of an amount of the environmental parameter of the soil substrate.

9. The soil sensor of claim 8 further comprising an onboard controller in signal communication with the output of the comparator module and configured to determine the amount of the environmental parameter of the soil substrate from the output signal.

10. The soil sensor of claim 9 wherein:
the sensing circuit further comprises an enable control module in signal communication with the signal generator;
the onboard controller is in signal communication with the enable control module and configured to generate a control signal for the enable control module; and
the enable control module selectively activates the signal generator in response to the control signal from the onboard controller.

11. The soil sensor of claim 10 wherein the enable control module is in signal communication with the comparator module and selectively activates the comparator module together with the signal generator in response to the control signal from the onboard controller.

12. The soil sensor of claim 8 wherein the comparator module is configured to determine a phase difference between the reference signal and the reflected signal and the output signal is based upon the phase difference.

13. The soil sensor of claim 12 wherein the comparator module comprises an XOR gate.

14. The soil sensor of claim 8 wherein the signal generator comprises an oscillator.

15. The soil sensor of claim 8 wherein:
the environmental parameter comprises volumetric water content;
the sensing module comprises a volumetric water content sensing module; and
the output signal facilitates determination of an amount of the volumetric water content in the soil substrate.

16. A method for determining a value of an environmental parameter of a soil substrate, the method comprising:
delivering a first reference signal into the soil substrate via a probe;
receiving a reflected signal by the probe that is generated as a function of propagation of the first reference signal through the soil substrate;
delivering a second reference signal to a comparator module that is in signal communication with the probe;
comparing, by the comparator module, the second reference signal with the reflected signal;
generating an output signal, by the comparator module, that is based upon the comparison of the second reference signal with the reflected signal; and
determining—an amount of the environmental parameter of the soil substrate based upon the output signal.

17. The method of claim 16 wherein the first reference signal and the second reference signal are substantially the same.

18. The method of claim 16 wherein the comparator module comprises an XOR gate.

19. The method of claim 16 wherein:
comparing the second reference signal with the reflected signal comprises determining a phase difference between the second reference signal and the reflected signal; and
generating the output signal comprises generating the output signal based upon the phase difference between the second reference signal and the reflected signal.

20. The method of claim 16 wherein delivering the first reference signal into the soil substrate further comprises periodically delivering the first reference signal into the soil substrate.
